(19) United States
(12) Patent Application Publication
(10) Pub. No.: US 2026/0235529 A1
(43) Pub. Date: Aug. 13, 2026

(54) AUTONOMOUS VISUAL INSPECTION SYSTEM FOR COBOTIC WELDING

(71) Applicant: L'Air Liquide, Societe Anonyme pour l'Etude et l’Exploitation des Procedes Georges Claude, Paris (FR)

(72) Inventors: Zongyao CHEN, Houston, TX (US); Charles CARISTAN, Houston, TX (US); Steven MIZE, Houston, TX (US)

(21) Appl. No.: 19/048,156

(22) Filed: Feb. 7, 2025

(51) Int. Cl. G01N 21/88; B25J 15/00; G01N 21/95; G01N 33/207; G06T 7/00; G06T 7/60

(52) U.S. Cl. CPC G01N 21/8851; B25J 15/0019; G01N 21/95; G01N 33/207; G06T 7/0004; G06T 7/60; G01N 2021/8887; G06T 2207/10024; G06T 2207/30164

(57) ABSTRACT

An autonomous visual inspection system for cobotic welding including an object to be welded, moving a robot welding arm to a first predetermined position, wherein a high-resolution camera acquires a first image of the object to be welded, using artificial intelligence to analyze the first image and determine if it qualifies for welding, performing a welding operation on the object to be welded, moving the robot welding arm to a second predetermined position, wherein the high-resolution camera acquires a second image, of the welded object, and using artificial intelligence to analyze the second image and determine if it a qualified weld.

### BACKGROUND

The increasing shortage of experienced welders has become an issue for manufacturing globally. Automated robotic welding plays an essential role in most large manufacturing companies. The environments in which traditional welding robots operate are unsafe for humans during operation. Therefore, programming these systems must be accomplished through a teaching pendant. The safety equipment, such as a pre-engineered work cell, is expensive, typically costing at least $50,000.

Collaborative robots or ‘cobots’ are robot arms that work with human beings. The cobot system takes less setup time than a robot welding system. The human operators can adjust the cobot arm pose manually with their hands. The features like customizable stop time and stop distance limits in the cobot joints can ensure safety when cobots work with operators.

The Universal Robot is one of the most popular collaborative robots in the market. Commercially available cobots address the skilled labor shortage by allowing companies to “hire” easy-to-use automated welding labor through short or long-term rental or lease programs.

Hence, developing vision capacity is a major task to improve the intelligence of the existing cobot welding system and automatically correct human errors. The vision guide Universal Robot was developed for pin-picking and has been applied in the industry. However, the welding process needs much higher repeat accuracy than the pin-picking task. Currently, there is no cobot welding system with a vision sensor for inspection on the market.

Although a few companies, such as Servo Robot and Binzel, developed laser-based guided vision systems for welding seam finding and tracking. These are also expensive, with the cost of each unit being more than $40K.

In this invention, we developed an intelligent vision-guided cobotic welding system, using artificial intelligence (AI), that can automatically inspect the weld part before and after welding.

### SUMMARY

An autonomous visual inspection system for cobotic welding including an object to be welded, moving a robot welding arm to a first predetermined position, wherein a high-resolution camera acquires a first image of the object to be welded, using artificial intelligence to analyze the first image and determine if it qualifies for welding, performing a welding operation on the object to be welded, moving the robot welding arm to a second predetermined position, wherein the high-resolution camera acquires a second image, of the welded object, and using artificial intelligence to analyze the second image and determine if it a qualified weld.

### BRIEF DESCRIPTION OF THE FIGURES

For a further understanding of the nature and objects for the present invention, reference should be made to the following detailed description, taken in conjunction with the accompanying drawings, in which like elements are given the same or analogous reference numbers and wherein:

FIG. 1 is a schematic representation of a typical cobot welding cell in accordance with one embodiment of the present invention

FIG. 2a is a schematic representation of the components of robot arm, in accordance with one embodiment of the present invention.

FIG. 2b is a schematic representation of the components of robot arm, in accordance with one embodiment of the present invention.

FIG. 3a is a schematic representation of the basic movements of robot arm, in accordance with one embodiment of the present invention.

FIG. 3b is a schematic representation of the basic movements of robot arm, in accordance with one embodiment of the present invention.

FIG. 4 is a schematic representation of the vision sensing device, in accordance with one embodiment of the present invention.

FIG. 5 is a schematic representation of a cobot welding cell, in accordance with one embodiment of the present invention.

FIG. 6 is a schematic representation of an object to be welded, illustrating the root opening, in accordance with one embodiment of the present invention.

FIG. 7 is a schematic representation of an object to be welded, illustrating the finished weld, in accordance with one embodiment of the present invention.

### ELEMENT NUMBERS

101=Control System
102=power source
103=robot arm
104=worktable
105=item to be welded
106=power source interface communication
107=hose package
108=robot arm interface communication cable
109=computer
201=base plate (of robot arm)
202=shoulder (of robot arm)
203=shoulder joint (of robot arm)
204=upper arm (of robot arm)
205=elbow joint (of robot arm)
206=lower arm (of robot arm)
207=wrist joint (of robot arm)
208=wrist (of robot arm)
209=welding torch
210=weld wire holder
211=weld wire conduit
212=wire feeder
213=torch cable
214=vision sensing device
301=first axis
302=second axis
303=third axis
304=fourth axis
305=fifth axis
306=sixth axis
401=camera
402=camera lens
403=polarizing filter
404=light source
405=camera shell
406=bandpass filter
501=vision sensing interface communication cable
601=root opening
602=width of the root opening
701=welding joint
702=width of the finished weld

### Description of Preferred Embodiments

Illustrative embodiments of the invention are described below. While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

As used herein, the term “artificial intelligence” is defined as software that is able to perceive the environment, learn and adapt, and take actions to maximize the possibility of best achieving a defined goal.

As used herein, the term “machine learning” is defined as a subset of artificial intelligence wherein a particular machine learns, adapts, and makes improvements to maximize achieving a defined goal, without additional external programming.

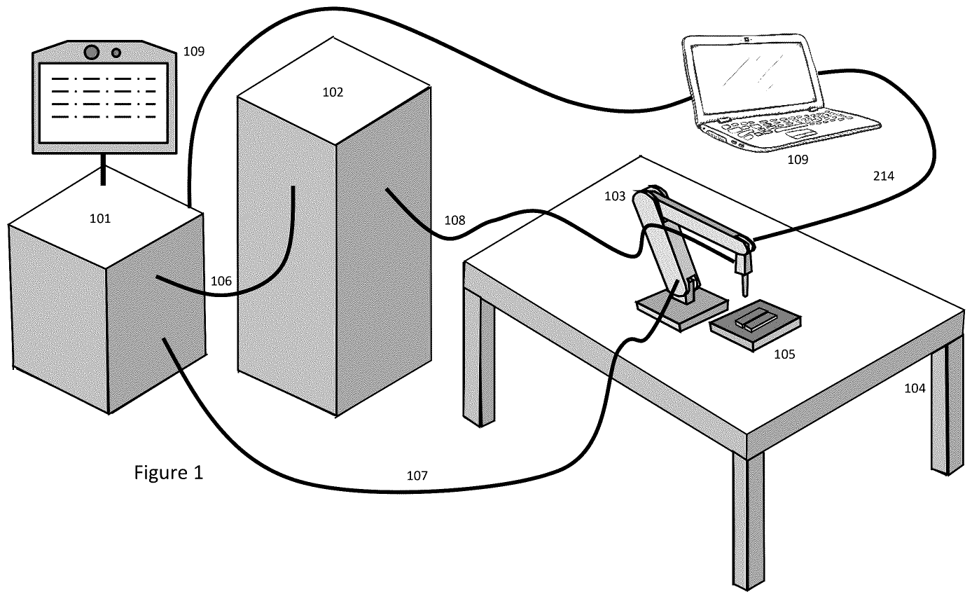

FIG. 1 illustrates a typical cobot welding cell in accordance with one embodiment of the present invention. Control system 101 controls both power source 102 and robot arm 103 to make them work simultaneously in the frame of a welding strategy. Control system 101 controls the trajectory of robot arm 103 and power source 102 controls the welding parameters (amperage, voltage, wire feeding speed). Power source 102 controls all consumables (gas and wire). Robot arm 103 will typically be attached to a worktable or bench 104, whereupon item 105 to be welded will be positioned. Control system 101 is functionally connected to power source 102 my means of power source interface communication cable 106. Power source 102 is functionally connected to robot arm 103 by means of hose package 107. Control system 101 may be functionally connected to robot arm 103 by means of robot arm interface communication cable 108. The cobot can be summarized as a high-end torch handler with all safety features (interlocks) embedded. The operator can use mobile devices such as smartphones to program the moving pass instead of using the original teaching pendant. Computer 109 is functionally connected to camera 214 and control system 101. Computer 109 may be a personal computer, an industrial computer, or any appropriate system known in the art.

Control system 101 may include a human machine interface (HMI) dashboard which may be utilized to visualize the actual cobot activity, or to visualize trends, measurements, etc. In one embodiment, the image data and calculated results may be uploaded to the cloud, through the human machine interface.

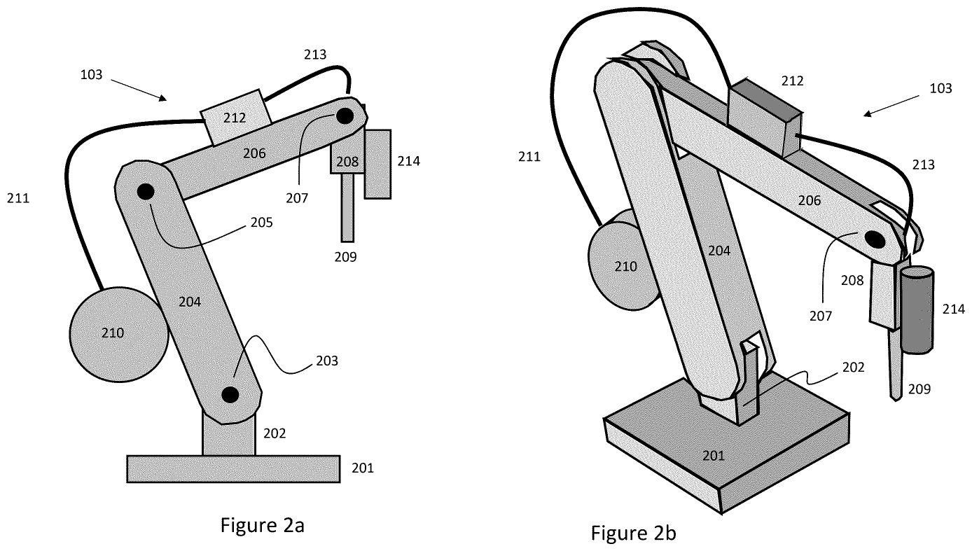

FIGS. 2a and 2b illustrate the components of robot arm 103 in accordance with one embodiment of the current invention. Base plate (sometimes referred to as the waist) 201 is affixed to the worktable or workbench (not shown), and to shoulder 202. Shoulder 202 is attached to upper arm 204 at shoulder joint 203. Upper arm 204 is attached to lower arm 206 at elbow joint 205. Lower arm 206 is attached to wrist 208 at wrist joint 207. Welding torch 209 is attached to wrist 208. Weld wire holder 210 may be attached to upper arm 204, or to some other location that is functionally acceptable. Weld wire conduit 211 is located between weld wire holder 210 and wire feeder 212 and provides the conduit for the wire to travel to the feeder. Weld wire conduit 211 passes through wire feeder 212 and is typically then referred to as torch cable 213. Weld wire 211 and torch cable 213 are the same cable. Torch cable (sometimes referred to as a whip) 213 connects welding torch 209 with weld wire holder 210 and provides wire to the torch. Vision sensing device 214 may be attached to lower arm 206, to wrist 208, or welding torch 209 facing the working bench (not shown)

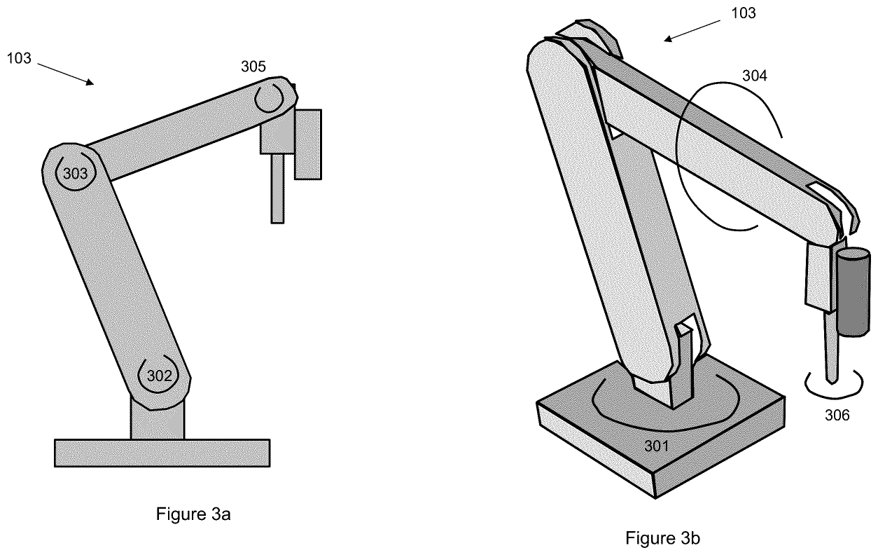

FIGS. 3a and 3b illustrate the basic movements of robot arm 103. Shoulder 202 may spin relative to base plate 201 on first axis 301. Upper arm 204 may pivot relative to shoulder 202 on second axis 302. Lower arm 206 may pivot relative to upper arm 204 on third axis 303. Lower arm 206 may spin relative to upper arm 204 on fourth axis 304. Wrist 208 may pivot relative to lower arm 206 on fifth axis 305. Welding torch 209 may spin relative to lower arm 206 on sixth axis 306.

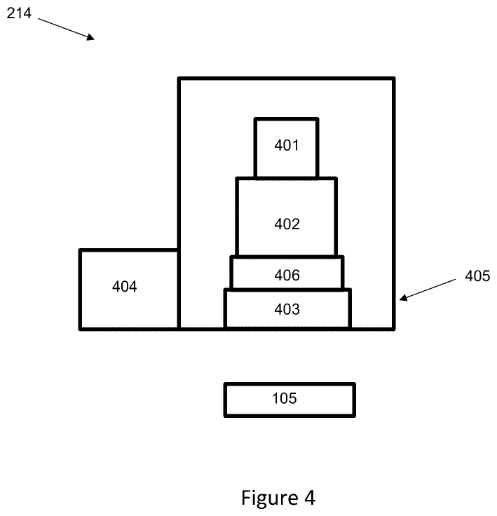

FIG. 4 illustrates the component of vision sensing device 214. Camera (and lens) 401 is located inside camera shell 405. Camera 401 may be a digital camera designed to capture and process a two-dimensional map of reflected intensity or contrast. Camera 401 may be used to evaluate the color, size, shape or location of item 105 to be welded. Camera shell 405 is designed to protect the vision sensors camera 401 and lens 402 from spatters and welding fumes during operation. Camera 401 is connected to control system 101 through an ethernet cable (not shown).

Light source 404 may be added to the outside of camera shell 405 and may be able to vary colors. A typical machine vision system utilizes ambient, white light. This is not always ideal but is obviously readily available. Multi-wavelength (RGB) lights may be used to facilitate optimal contrast and visibility. Light source 404 may have multi-color selectivity. In some cases, a red source, such as a red LED, may be best as they often correspond with the peak sensitivity of the camera's sensor. Polarizing filter 403 may be added if necessary. A bandpass filter 406 may be added in between lens 402 and polarizing filter 403 to reduce the ambient light.

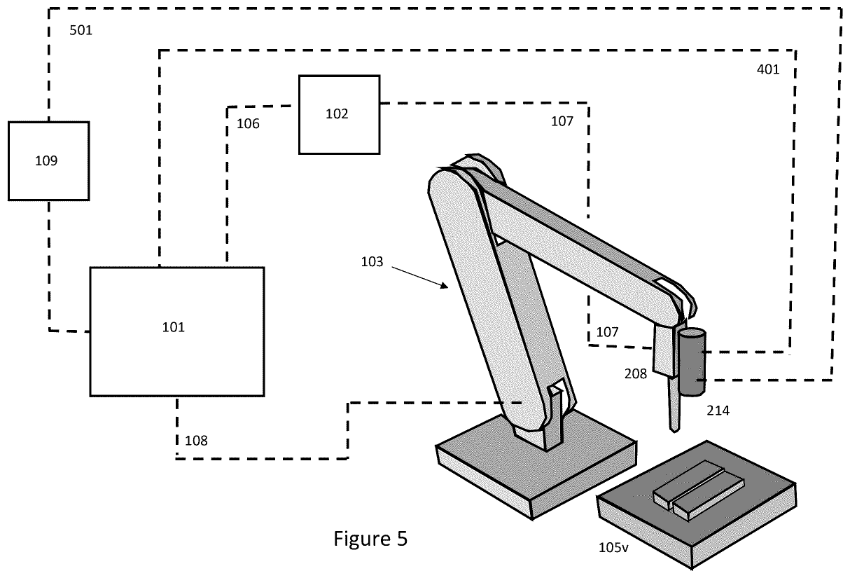

FIG. 5 illustrates a cobot welding cell in accordance with the current invention. Control system 101 controls both power source 102 and robot arm 103. Control system 101 is functionally connected to power source 102 my means of power source interface communication cable 106. Power source 102 is functionally connected to robot arm 103 by means of hose package 107. Control system 101 may be functionally connected to robot arm 103 by means of robot arm interface communication cable 108. Computer 109 may be functionally connected to vision sensing device 214 by means of vision sensing interface communication cable 501. Computer 109 may be functionally connected to the control system 101.

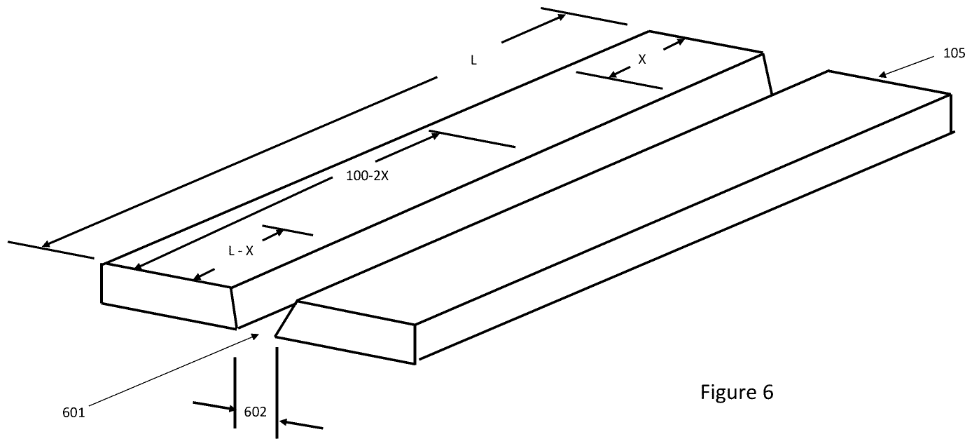

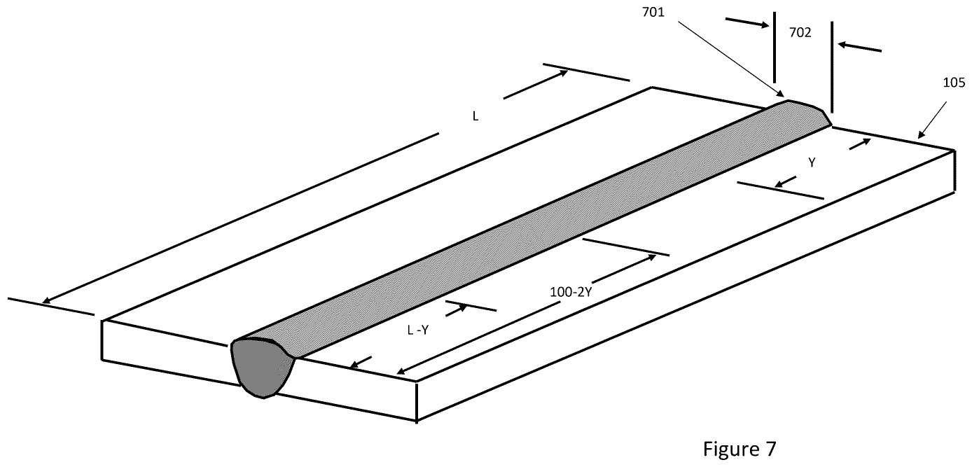

FIGS. 6 and 7 illustrate the identification of the root opening 601 and the welding joint 701 in accordance with one embodiment of the present invention. The width of the root opening 602 may be measured. This may include, but is not limited to, the average width, the minimum width, the maximum width, the median width, and the standard deviation, as are known in the art. The width of the root opening 602 may be measured at more than one location, preferably at three different locations. The locations may be the first x % of the segment, the last x % of the segment, and an intermediate point (between 100-2 x %). Wherein “the first x % of the segment” is defined as (distance X/overall length L)×100. Wherein “the last x % of the segment” is defined as (distance L−X/overall length L)×100.

Various types of joints known in the art (and not illustrated herein) include, but are not limited to, tee joints, lap joints, and square butt joints. These may also include linear and circumferential joints. In some embodiments, the root opening and the width of the prepared V-shape butt joint can be measured.

The width of the finished weld 702 may be measured. This may include, but is not limited to, the average width, the minimum width, the maximum width, the median width, and the standard deviation, as are known in the art. The width of the weld 702 may be measured at more than one location, preferably at three different locations. The locations may be the first y % of the segment, the last y % of the segment, and an intermediate point (between 100-2 y %). Wherein “the first y % of the segment” is defined as (distance y/overall length L)×100. Wherein “the last y % of the segment” is defined as (distance L−y/overall length L)×100. In one embodiment, x is approximately equal to y.

Various types of joints known in the art (and not illustrated herein) include, but are not limited to, tee joints, lap joints, and square butt joints. These may also include linear and circumferential joints. In some embodiments, the root opening and the width of the prepared V-shape butt joint can be measured.

The main procedures for welding inspection with a vision-guided cobot welding system include four steps:
1. Robot programming. The user can program the robot path with a teaching pendant (not shown) or by using a mobile device such as a smart phone. The moving path may include moves in the air and welding motions.
2. Pre-welding image acquisition and assessment. The cobot arm is intended to move to a predetermined position (or positions) which is close to the welding object. From this position, the cobot arm takes a first image of the part, before welding. The artificial intelligence (AI) software will then calculate the geometry and dimensional signature of the part and width of the welding joint gap. The artificial intelligence (AI) software will then determine if the parts qualify for welding.

The artificial intelligence software may compare the measured results with an expected value of the welded joints, gaps, and weld seams and notify the user prior to welding. An estimated consumption of welding wire may also be made. The artificial intelligence software may identify the relevant boundaries of the workpiece, and, if necessary, calculate the overall size of the workpiece. The artificial intelligence software may compare the measured results with the expected size of the workpiece, and notify the user prior to welding. The artificial intelligence software may also recognize one or more corners of the work piece, and may be used to represent a dimensional signature of the work piece. The artificial intelligence software may output to a human machine interface. The human machine interface may output to the cloud.
3. Welding. The cobot welding system executes the programmed welding path and completes the welding process.
4. Post-welding image acquisition and assessment. The cobot moves its arm to a predetermined position close to the welded part and captures a second image of the part, after welding. The artificial intelligence (AI) software calculates the weld seam geometry, calculates the material usage of the weld, and determines if it is qualified.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. An autonomous visual inspection system for cobotic welding, comprising;
an object to be welded,
moving a robot welding arm to a first predetermined position, wherein a high-resolution
camera acquires a first image of the object to be welded,
using artificial intelligence to analyze the first image and determine if it qualifies for welding,
performing a welding operation on the object to be welded,
moving the robot welding arm to a second predetermined position, wherein the high-resolution camera acquires a second image, of the welded object, and
using artificial intelligence to analyze the second image and determine if it a qualified weld.

2. The system of claim 1, wherein the first predetermined position and the second predetermined position may not be the same.

3. The system of claim 1, wherein the object to be welded comprises a root opening, and wherein the root opening is measured, the artificial intelligence compares the root opening measurement with an expected value and notifies an operator of a variance.

4. The system of claim 1, wherein the object to be welded has boundaries, wherein the artificial intelligence identifies the boundaries, calculates the size of the object to be welded, compares the calculated size of the object to be welded to an expected size, and notifies an operator of a variance.

5. The system of claim 1, wherein the object to be welded comprises a finished weld, and wherein the weld is measured, the artificial intelligence compares the weld measurement with an expected value and notifies an operator of a variance.

6. The system of claim 1, wherein the object to be welded has corners, wherein the artificial intelligence identifies the corners, calculates distance between the corners, and calculates a dimensional signature of the workpiece.

7. The system of claim 1, further comprising a human machine interface dashboard, wherein an operator receives an image of the object to be welded, receives the analysis of the first image, and receives the analysis of the second image.

8. The system of claim 7, wherein the image of the object to be welded, the analysis of the first image, and the analysis of the second image are uploaded from the human machine interface dashboard to a data cloud.

9. The system of claim 1, wherein the high-resolution camera utilizes a multi-color light source configured to have multi-color selectivity, and further comprises a means of dust and welding fume protection, configured to protect the high-resolution camera and multi-color light source during a welding operation.
